(19) United States
(12) Patent Application Publication
(10) Pub. No.: US 2022/0184293 A1
(43) Pub. Date: Jun. 16, 2022

(54) GAS DELIVERY SYSTEM

(71) Applicant: Mallinckrodt Pharmaceuticals Ireland Limited, Dublin (IE)

(72) Inventor: James Potenziano, Hazelwood, MO (US)

(21) Appl. No.: 17/598,058

(22) PCT Filed: Mar. 25, 2020
(86) PCT No.: PCT/US2020/024632

§ 371 Date: Sep. 24, 2021

Related U.S. Application Data
(60) Provisional application No. 62/823,297, filed on Mar. 25, 2019.

(51) Int. Cl. A61M 1/36; A61M 60/113; A61M 60/38; A61M 1/16; A61M 60/232

(52) U.S. Cl. CPC A61M 1/3687; A61M 1/3666; A61M 1/3613; A61M 60/113; A61M 2202/0291; A61M 1/1698; A61M 60/232; A61M 1/1601; A61M 60/38

(57) ABSTRACT

A gas delivery system is provided. The gas delivery system includes a pump operable to receive blood from a patient. A gas transfer unit is in fluid communication with the pump and operable to receive the blood from the pump and deliver a therapeutic amount of xenon gas to the blood. A patient connector withdraws and/or infuses the blood into the patient.

### CROSS-REFERENCE TO RELATED APPLICATIONS

This application claims priority to U.S. Provisional Application No. 62/823,297, filed Mar. 25, 2019, the entire contents of which are hereby incorporated by reference.

### FIELD

The present disclosure relates generally to systems and methods to deliver therapeutic gases to withdrawn blood. In at least one example, the present disclosure relates to system and methods to therapeutically deliver xenon gas to the withdrawn blood of patients at risk of reperfusion injury.

### BACKGROUND

Reperfusion injury includes tissue damage when blood supply returns to the tissue after a period of lack of oxygen. Reperfusion injury can occur, for example, after a stroke, cardiac arrest, and/or traumatic brain injury. Noble gases, such as xenon and/or argon, can help reduce damage from reperfusion injury.

### BRIEF DESCRIPTION OF THE DRAWINGS

Implementations of the present technology will now be described, by way of example only, with reference to the attached figures, wherein:

FIG. 1 is a diagram of a system to deliver therapeutic nitric oxide to a patient according to the present disclosure;

FIG. 2A is a diagram of an exemplary configuration of the system;

FIG. 2B is a diagram of another exemplary configuration of the system;

FIG. 2C is a diagram of another exemplary configuration of the system;

FIG. 3 is a diagram of an exemplary system to deliver therapeutic nitric oxide to a patient;

FIG. 4 is a block diagram of an exemplary controller; and

FIG. 5 is a flowchart of an exemplary method of preventing or inhibiting reperfusion injury in a patient.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented. The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. For example, “substantially cylindrical” means that the object resembles a cylinder, but can have one or more deviations from a true cylinder. The terms “comprising,” “including” and “having” are used interchangeably in this disclosure. The terms “comprising,” “including” and “having” mean to include, but not necessarily be limited to the things so described.

Disclosed herein is a gas delivery system to deliver one or more therapeutic gases, such as xenon gas, to a patient with ischemia. The therapeutic gas can be delivered directly to the blood instead of introducing the gas through the lungs. The lungs have inefficiencies in processing gas, so there may be waste in gas that is not processed. Also, the amount of gas that is processed is not consistently predictable, so delivering/dissolving gas directly into the blood and bypassing the lungs can more accurately assure the concentration of gas in the blood.

Additionally, to introduce gas through inhalation from the lungs, the patient must be intubated and/or sedated. By bypassing the lungs and delivering the gas directly into the bloodstream, the patient can be awake and/or non-intubated and may be treated in an ambulatory setting. For example, if the patient experienced a debilitating event such as a stroke, cardiac arrest, and/or traumatic brain injury, the patient may have experienced a period where tissues lacked oxygen. When the blood supply returns to the tissue after a period of lack of oxygen, reperfusion injury can occur, resulting in detrimental effects.

Introduction of xenon gas has been shown to reduce the damage from reperfusion injury. In some examples, noble gases such as argon can also be introduced into the subject to help reduce the damage from reperfusion injury. Additionally, in some examples, oxygen can be introduced into the blood if the patient is not able to adequately breathe. To more effectively introduce the one or more therapeutic gases into the patient, a gas delivery system can be utilized to directly deliver the therapeutic gases into the blood stream of the patient.

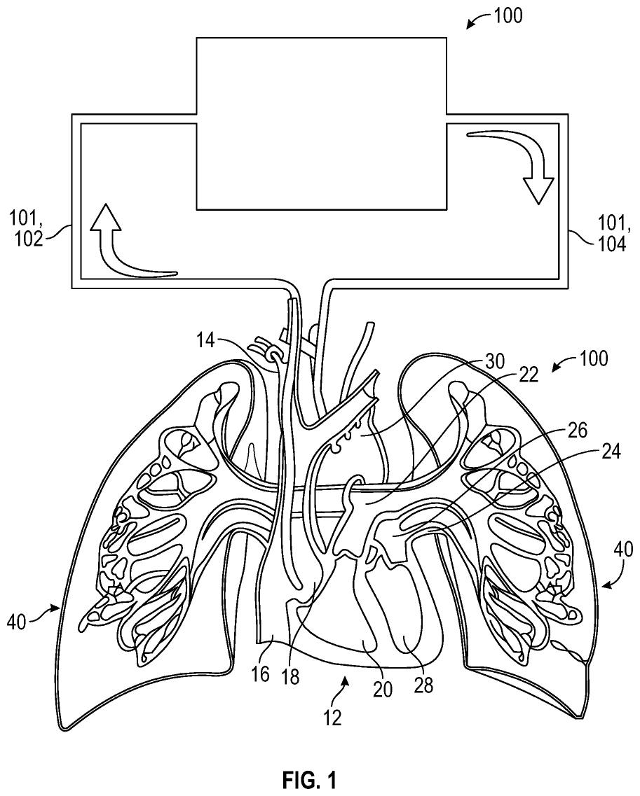

The gas delivery system can be utilized with a patient as shown, for example, in FIG. 1. The gas delivery system 100 is operable to deliver, for example dissolve, one or more therapeutic gases to a patient 10. For example, the gas delivery system 100 can deliver therapeutic xenon gas, argon gas, and/or oxygen gas to a patient 10, for example a patient that may be subject to reperfusion injury. The therapeutic gas can be solubilized into the blood. The gas delivery system 100 can include a patient connector 101 connected with the patient 10 and operable to withdraw and/or infuse blood into the patient 10. In some examples, the patient connector 101 can include a first conduit 102 and a second conduit 104. The first conduit 102 and the second conduit 104 can be, for example, cannulas. The first conduit 102 and the second conduit 104 can be inserted into the circulatory system of the patient 10.

As there can be variability in respiration rates and efficiency of the lungs, it can be difficult to deliver a constant therapeutic amount of therapeutic gases to the patient 10 by inhalation. Additionally, gases solubilized in blood may be breathed out by the patient and/or taken up by tissue, so the concentration of therapeutic gas in the blood may be inconsistent. Accordingly, bypassing the lungs 40 can better insure the therapeutic amount of therapeutic gas is solubilized in the blood and delivered to the patient 10. To bypass the lungs 40, as illustrated in FIG. 1, the first conduit 102 can be inserted into the right atrium 18 to pump blood from the patient 10 through the gas delivery system 100. After passing the fluid through the gas delivery system 100, the fluid solubilized with the one or more therapeutic gases can be pumped back into the body of the patient. The second conduit 104 can be inserted into the aorta 30 such that the blood is pumped away from the heart 12 to the rest of the patient 10.

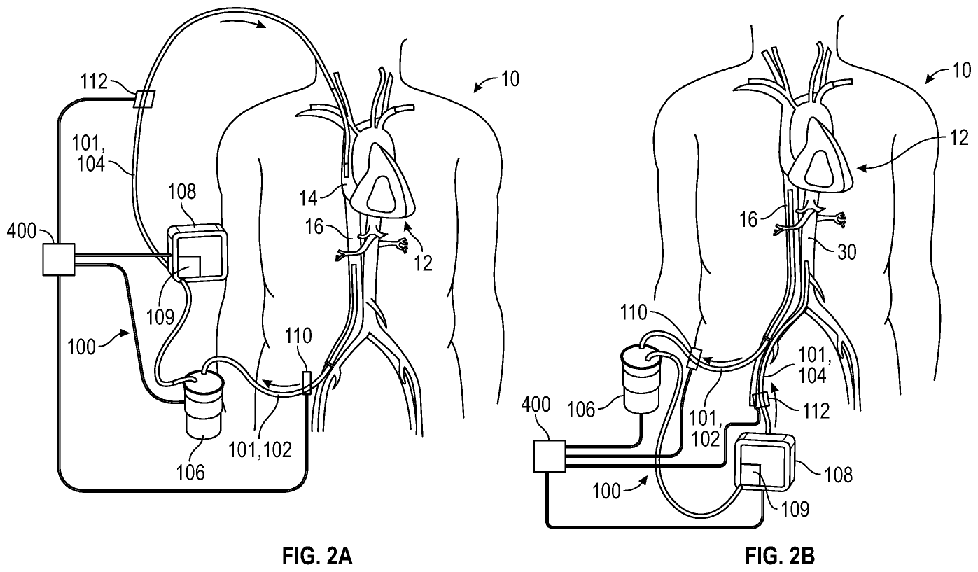

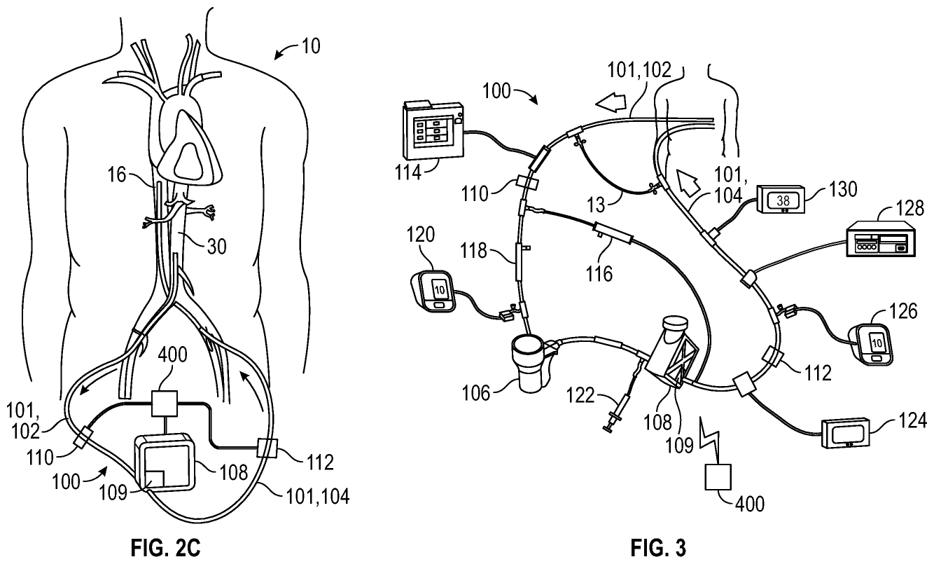

FIGS. 2A-3 illustrate different examples of configurations of the gas delivery system 100 in fluid connection with the patient 10. While FIGS. 2A-3 illustrate the components of the gas delivery system 100 as separate and independent components, in at least one example, at least one of the components of the gas delivery system 100 can be contained within one or more housings (not shown). In some examples, one or more of the components of the gas delivery system 100 can be removably coupled within the gas delivery system 100 to allow for easy replacement and/or cleaning.

The gas delivery system 100 can include a pump 106 and a gas transfer unit 108. The gas transfer unit 108 is operable to dissolve a therapeutic amount of one or more therapeutic gases in a fluid such as blood. For example, the gas transfer unit 108 can be operable to deliver a therapeutic amount of xenon gas to blood. In at least one example, the therapeutic amount of solubilized xenon in the blood may be about two times the solubilized amount of oxygen in the blood. In another example, the partial pressure of xenon in the blood may be about two times the partial pressure of oxygen (PaO₂). In some examples, the gas transfer unit 108 can also deliver a predetermined amount of oxygen gas to the blood. In some examples, the gas transfer unit 108 can include a gas exchange membrane 109 through which gas can be exchanged and/or delivered to the blood. In some examples, the gas transfer unit 108 can be included in an extracorporeal membrane oxygenation (ECMO) system. In some examples, the gas transfer unit 108 can be included in an ambulatory ECMO system. The pump 106 is operable to pump the fluid from the patient 10 through the gas transfer unit 108 and back to the patient 10. In at least one example, the pump 106 can be a centrifugal pump. The first conduit 102 can be in fluidic communication with the pump 106 and the gas transfer unit 108 and operable to be inserted into the patient 10 to withdraw the blood. The second conduit 104 can be in fluidic communication with the pump 106 and the gas transfer unit 108 and be operable to be inserted into the patient 10 such that the blood is infused back into the patient 10.

As illustrated in FIG. 2A-2C, the first conduit 102 and the second conduit 104 can be inserted into the patient 10. While FIGS. 2A-2C illustrate exemplary configurations, the system 100 is not limited to the illustrated configurations. Additionally, the configurations may be adjusted depending on the situation and/or the patient. For example, an infant may require a different configuration than an adult. An ambulatory setting may also require a different configuration than in a hospital setting.

In at least one example, the first conduit 102 can be inserted into and be in fluidic communication with a vein of the patient 10, and the second conduit can be inserted into and be in fluidic communication with an artery of the patient 10. As illustrated in FIG. 2A, the first conduit 102 is inserted into the inferior vena cava, and the second conduit 104 can be inserted into the right atrium. As illustrated in FIG. 2B, the first conduit 102 can be inserted into the inferior vena cava, and the second conduit 104 can be inserted into the aorta. In other examples, the first conduit 102 can be inserted into an artery and the second conduit 104 can be inserted into a vein. As illustrated in FIG. 2C, the first conduit 102 can be inserted into the aorta, and the second conduit 104 can be inserted into the inferior vena cava. As illustrated in FIG. 2C, the gas delivery system 100 may not include a pump 106, relying on the heart 12 to pump the fluid through the gas delivery system 100. In at least one example, the first conduit 102 and/or the second conduit 104 can be inserted into the patient 10 through the femoral vein and/or the femoral artery. In some examples, the first conduit 102 and/or the second conduit 104 can be inserted into the patient 10 through the jugular vein.

The gas delivery system 100 may provide a consistent delivery of xenon gas to a patient by continuous monitoring of the amount of xenon gas within the blood prior to and after delivery of the xenon gas to the blood. Solubilized xenon gas in blood may be breathed out by the patient and/or taken up by tissue, so the concentration of therapeutic gas in the blood may be inconsistent. Therefore, the gas delivery system 100 may automatically adjust the amount of xenon gas delivered in the gas transfer unit 108 based on feedback from at least one gas detection sensor. In some examples, the gas delivery system 100 can include a first gas detection sensor 110 and/or a second gas detection sensor 112. The first gas detection sensor 110 is located upstream from the gas transfer unit 108 such that the first gas detection sensor 110 is disposed between the patient 10 and the gas transfer unit 108 along the first conduit 102. The first gas detection sensor 110 is operable to measure a first concentration of the therapeutic gas in the blood prior to passing through the gas transfer unit 108. For example, the first gas detection sensor 110 can measure a first concentration of xenon in blood from the patient 10 prior to gas delivery by the gas transfer unit 108. In some examples, the first gas detection sensor 110 can measure the solubilized concentration of xenon. In at least one example, as illustrated in FIGS. 2A-3, the first gas detection sensor 110 can be located upstream from the pump 106 to determine the amount of therapeutic gas that was expelled by the patient 10. In some examples, the first gas detection sensor 110 can be located downstream from the pump 106 but upstream from the gas transfer unit 108 to also account for the amount of therapeutic gas that may have been expelled from the blood due to the pump 106. In some examples, the first gas detection sensor 110 can be located upstream from the pump 106, and an additional sensor can be located downstream from the pump 106 and upstream from the gas transfer unit 108 such that the amount of therapeutic gas lost due to the pump 106 can be determined. It can be determined whether the pump 106 may need to be replaced and/or if there are inefficiencies within the system 100.

The second gas detection sensor 112 is located downstream from the gas transfer unit 108 and operable to measure a second concentration of therapeutic gas in the fluid after passing through the gas transfer unit 108. For example, the second gas detection sensor 112 can measure a second concentration of xenon in the blood after passing through the gas transfer unit 108. In some examples, the second gas detection sensor 112 can measure the solubilized concentration of xenon. Accordingly, the second gas detection sensor 112 can detect and determine the concentration of xenon being delivered to the patient 10.

Measuring additional gases in the blood may further inform a user how the patient's body is functioning and further inform adjustments to the delivery of the therapeutic gas to the patient. The gas delivery system 100 may further include additional gas detection sensors operable for measuring concentrations of additional gases in the blood. For example, the gas delivery system 100 may include an oxygen gas detection sensor and/or a carbon dioxide gas detection sensor at any point along the first conduit 102 or second conduit 104. In some examples the first gas detection sensor 110 and/or the second gas detection sensor 112 may be further operable to measure a concentration of oxygen and/or carbon dioxide.

The gas delivery system 100 includes a controller 400 communicatively coupled with the pump 106, the gas transfer unit 108, the first gas detection sensor 110, and/or the second gas detection sensor 112. In some examples, the controller 400 may be communicatively coupled to any additional gas detection sensors. Features of the controller 400 will be discussed in further detail in FIG. 4. The controller 400 can be coupled with components of the gas delivery system 100 by any suitable wired or wireless connection, for example Ethernet, Bluetooth, RFID, and/or fiber optic cable. In at least one example, the controller 400 is contained within a housing with the components of the gas delivery system 100. In some examples, the controller 400 can be separate and independent from the components of the gas delivery system 100.

In at least one example, the controller 400 can receive the first concentration of therapeutic gas from the first gas detection sensor 110. The second concentration of the therapeutic gas can be received by the controller 400 from the second gas detection sensor 112. The controller 400 compares the first concentration with the second concentration, and when the first concentration is less than the second concentration, the controller 400 can determine an additional amount of therapeutic gas to be delivered to the fluid by the gas transfer unit 108 based on the first concentration of therapeutic gas and the second concentration of the therapeutic gas. In some examples, the controller 400 can determine the additional amount of therapeutic gas automatically without any human interaction or assistance. The controller 400 can then adjust, when the first concentration is less than the second concentration, the gas transfer unit 108 to deliver the additional amount of therapeutic gas to the blood such that the blood includes the therapeutic amount of therapeutic gas. For example, the second concentration of xenon may correlate with the desired and predetermined therapeutic amount of xenon to be delivered to the blood. As xenon gas is a noble gas, the body should not metabolize the xenon gas, so the first concentration of xenon gas should be the same as the second concentration of xenon gas after the xenon gas has been first delivered to the patient's blood. However, xenon gas may be exhaled by the patient and/or absorbed by some tissues. To ensure that the correct amount of xenon is delivered to the body, when the first concentration is less than the second concentration, the controller 400 can determine the additional amount of therapeutic gas needed and adjust the gas transfer unit 108 to deliver the additional amount of xenon gas to the blood.

In at least one example, the controller 400 can determine, by the second concentration from the second gas detection sensor 112, whether the gas transfer unit 108 delivered the correct amount of the therapeutic gas to the blood. For example, if the second concentration from the second gas detection sensor 112 is less than the therapeutic amount, the gas transfer unit 108 may need to be replaced and/or recalibrated. In some examples, the controller 400 can increase the flow rate and/or adjust the gas delivery system 100 to ensure that the desired amount of therapeutic gas is delivered to the blood.

As a safety measure, the amount of additional gases in the blood, such as oxygen and carbon dioxide, may be used to confirm the gas delivery system 100 is functioning properly. In an example, the gas delivery system 100 may further include an automatic shutoff valve in communication with the controller 400. In at least one example, the controller 400 may receive a concentration of oxygen or carbon dioxide from an additional gas detection sensor. The controller 400 may then compare the concentration of oxygen or carbon dioxide to a threshold level of oxygen or carbon dioxide set by the user and close the automatic shutoff valve if the concentration of oxygen or carbon dioxide is outside the threshold level.

FIG. 3 illustrates an exemplary gas delivery system 100 which includes the first conduit 102, the second conduit 104, the pump 106, the gas transfer unit 108, the first gas detection sensor 110, and/or the second gas detection sensor 112 as discussed above. The gas delivery system 100 can include any combination of the components illustrated in FIG. 3. In some examples, the gas delivery system 100 can include an ECMO system.

A bridge 13 can fluidly connect the first conduit 102 and the second conduit 104. The bridge 13 can include one or more valves to permit or restrict the blood to pass across the bridge 13, bypassing the rest of the gas delivery system 100. A venous saturation monitor 114 disposed upstream of the gas transfer unit 108 can determine the oxygen saturation of the withdrawn blood. A bladder 118 can control the suction of the blood from the patient from the pump 106. The bladder 118 prevents continuing suction when the first conduit 102 is occluded, for example, for more than a few seconds. A first pressure monitor 120 can be located upstream of the pump 106 and downstream of the bladder 118 and can monitor the pressure within the first conduit 102. A hemofilter 116 fluidly connect the first conduit 102 and the second conduit 104 such that the pump 106 and/or the gas transfer unit 108 are bypassed. The blood can be passed through the hemofilter 116 to remove waste products and water. The first conduit 102 can form one or more ports 122 which permit retrieval of the blood and/or delivery of components into the blood. For example, heparin can be injected into the blood through a port 122. In some examples, samples can be retrieved through the port 122.

A bubble sensor 124 can be located downstream from the gas transfer unit 108 and operable to detect air bubbles in the blood. The bubble sensor 124 can include an alarm to signal to a user that bubbles are detected in the blood. The alarm can be an audible alarm, a visual alarm, and/or a mechanical alarm such as vibration. A second pressure monitor 126 can be disposed downstream from the gas transfer unit 108 and can monitor the pressure within the second conduit 104. A flow meter 128 can measure and monitor the flow of the blood through the second conduit 104 to the patient 10. In at least one example, the flow meter 128 can include a transonic flow meter. A temperature unit 130 is operable to measure, monitor, and/or maintain the blood at a predetermined temperature. For example, the temperature unit 130 can include a temperature exchange system which transfers heat to the blood and/or removes heat from the blood such that the blood is maintained within a predetermined temperature range, for example standard body temperature.

FIG. 4 is a block diagram of an exemplary controller 400. Controller 400 is configured to perform processing of data and communicate with one or more components of the gas delivery system 100, for example as illustrated in FIGS. 1-3. In operation, controller 400 communicates with one or more of the above-discussed components and may also be configured to communication with remote devices/systems.

As shown, controller 400 includes hardware and software components such as network interfaces 410, at least one processor 420, sensors 460 and a memory 440 interconnected by a system bus 450. Network interface(s) 410 can include mechanical, electrical, and signaling circuitry for communicating data over communication links, which may include wired or wireless communication links. Network interfaces 410 are configured to transmit and/or receive data using a variety of different communication protocols, as will be understood by those skilled in the art.

Processor 420 represents a digital signal processor (e.g., a microprocessor, a microcontroller, or a fixed-logic processor, etc.) configured to execute instructions or logic to perform tasks in a wellbore environment. Processor 420 may include a general purpose processor, special-purpose processor (where software instructions are incorporated into the processor), a state machine, application specific integrated circuit (ASIC), a programmable gate array (PGA) including a field PGA, an individual component, a distributed group of processors, and the like. Processor 420 typically operates in conjunction with shared or dedicated hardware, including but not limited to, hardware capable of executing software and hardware. For example, processor 420 may include elements or logic adapted to execute software programs and manipulate data structures 445, which may reside in memory 440.

Sensors 460, which may include first gas detection sensor 110 and/or second gas detection sensor 112 as disclosed herein, typically operate in conjunction with processor 420 to perform measurements, and can include special-purpose processors, detectors, transmitters, receivers, and the like. In this fashion, sensors 460 may include hardware/software for generating, transmitting, receiving, detection, logging, and/or sampling magnetic fields, seismic activity, and/or acoustic waves, temperature, pressure, or other parameters.

Memory 440 comprises a plurality of storage locations that are addressable by processor 420 for storing software programs and data structures 445 associated with the embodiments described herein. An operating system 442, portions of which may be typically resident in memory 440 and executed by processor 420, functionally organizes the device by, inter alia, invoking operations in support of software processes and/or services 444 executing on controller 400. These software processes and/or services 444 may perform processing of data and communication with controller 400, as described herein. Note that while process/service 444 is shown in centralized memory 440, some examples provide for these processes/services to be operated in a distributed computing network.

It will be apparent to those skilled in the art that other processor and memory types, including various computer-readable media, may be used to store and execute program instructions pertaining to the fluidic channel evaluation techniques described herein. Also, while the description illustrates various processes, it is expressly contemplated that various processes may be embodied as modules having portions of the process/service 444 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 420 or computer readable medium encoded with instructions for execution by processor 420 that, when executed by the processor, are operable to cause the processor to perform the functions described herein.

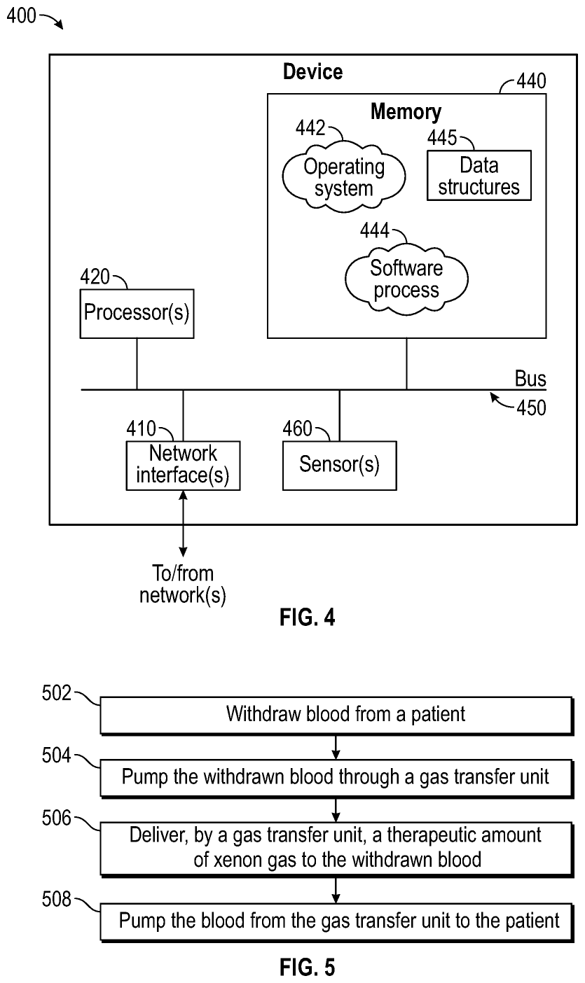

Referring to FIG. 5, a flowchart is presented in accordance with an example embodiment. The method 500 is provided by way of example, as there are a variety of ways to carry out the method. The method 500 described below can be carried out using the configurations illustrated in FIGS. 1-4, for example, and various elements of these figures are referenced in explaining example method 500. Each block shown in FIG. 5 represents one or more processes, methods or subroutines, carried out in the example method 500. Furthermore, the illustrated order of blocks is illustrative only and the order of the blocks can change according to the present disclosure. Additional blocks may be added or fewer blocks may be utilized, without departing from this disclosure.

The example method 500 is a method of preventing or inhibiting reperfusion injury in a patient. In at least one example, the patient may have ischemia. In some examples, the patient may be awake. In some examples, the patient may be non-intubated. Accordingly, with the method 500, the patient does not have to be intubated and/or sedated. The example method 500 can begin at block 502.

At block 502, blood is withdrawn from a patient. A first conduit, for example a cannula, can be inserted into the patient to withdraw the blood. In at least one example, the first conduit can be inserted into and be in fluidic communication with a vein of the patient. The blood can be withdrawn by a pump in fluidic communication with the first conduit, for example a centrifugal pump.

At block 504, the withdrawn blood is pumped through a gas transfer unit. A first gas detection sensor located upstream of the gas transfer unit can measure a first concentration of the xenon in the blood prior to passing through the gas transfer unit.

At block 506, the gas transfer unit delivers a therapeutic amount of xenon gas to the withdrawn blood. In at least one example, the gas transfer unit can also deliver a predetermined amount of oxygen gas to the blood. In some examples, the gas transfer unit can deliver one or more therapeutic gases to the blood when the blood passes through a gas exchange membrane. In some examples, the gas transfer unit can be included in an extracorporeal membrane oxygenation (ECMO) system.

A second gas detection sensor located downstream of the gas transfer unit can measure a second concentration of xenon in the blood after passing through the gas transfer unit. The second concentration may correspond with the therapeutic amount of xenon desired. A controller can be communicatively coupled with the first gas detection sensor, the second gas detection sensor, the pump, and/or the gas transfer unit. The controller can compare the first concentration and the second concentration. When the first concentration is less than the second concentration, the controller can determine an additional amount of xenon gas to be delivered to the blood. The controller can then adjust, when the first concentration is less than the second concentration, the gas transfer unit to deliver the additional amount of xenon to the blood such that the blood includes the therapeutic amount of xenon gas. In at least one example, the controller can automatically determine the additional amount of xenon gas to be delivered to the blood without human interaction or assistance.

At block 508, the blood is pumped from the gas transfer unit to the patient. The blood can be pumped through a second conduit which can be inserted into the patient to infuse the blood into the patient. In at least one example, the second conduit can include a cannula. In some examples, the second conduit can be inserted into and be in fluidic communication with an artery of the patient. In some examples, a temperature unit can measure and adjust the temperature of the blood such that the blood is maintained at a predetermined temperature.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A gas delivery system for delivering xenon gas to a patient, the gas delivery system comprising:
a pump operable to receive blood from a patient;
a gas transfer unit in fluid communication with the pump and operable to receive the blood from the pump and deliver a therapeutic amount of xenon gas to the blood; and
a patient connector operable to withdraw and/or infuse the blood into the patient.

2. The gas delivery system of claim 1, wherein the patient is awake.

3. The gas delivery system of claim 1, wherein the patient is non-intubated.

4. The gas delivery system of claim 1, further comprising:
a first gas detection sensor located upstream from the gas transfer unit and operable to measure a first concentration of the xenon in the blood prior to passing through the gas transfer unit; and
a second gas detection sensor located downstream from the gas transfer unit and operable to measure a second concentration of xenon in the blood after passing through the gas transfer unit.

5. The gas delivery system of claim 4, further comprising: a controller coupled with the gas transfer unit, the first gas detection sensor, and the second gas detection sensor, the controller being operable to:
receive the first concentration of xenon from the first gas detection sensor;
receive the second concentration of xenon from the second gas detection sensor;
compare the first concentration and the second concentration;
determine, when the first concentration is less than the second concentration gas, an additional amount of xenon gas to be delivered to the blood; and
adjust, when the first concentration is less than the second concentration, the gas transfer unit to deliver the additional amount of xenon to the blood such that the blood includes the therapeutic amount of xenon.

6. The gas delivery system of claim 5, wherein the controller automatically determines the additional amount of xenon gas to be delivered to the blood.

7. The gas delivery system of claim 1, wherein the gas transfer unit is further operable to deliver a predetermined amount of oxygen gas to the blood.

8. The gas delivery system of claim 1, wherein the gas transfer unit is included in an extracorporeal membrane oxygenation (ECMO) system.

9. The gas delivery system of claim 1, wherein the pump includes a centrifugal pump.

10. The gas delivery system of claim 1, wherein the patient connector further includes:
a first conduit in fluidic communication with the pump and the gas transfer unit operable to be inserted into the patient to withdraw the blood;
a second conduit in fluidic communication with the pump and the gas transfer unit operable to be inserted into the patient such that the blood is infused into the patient.

11. The gas delivery system of claim 10, wherein the first conduit is operable to be inserted into and be in fluidic communication with a vein of the patient, and the second conduit is operable to be inserted into and be in fluidic communication with an artery of the patient.

12. The gas delivery system of claim 1, further comprising:
a temperature unit operable to maintain the blood at a predetermined temperature.

13. A method of preventing or inhibiting reperfusion injury in a patient with ischemia, the method comprising:
withdrawing blood from a patient;
pumping the withdrawn blood through a gas transfer unit;
delivering, by the gas transfer unit, a therapeutic amount of xenon gas to the withdrawn blood; and
pumping the blood from the gas transfer unit to the patient.

14. The method of claim 13, wherein the patient is awake.

15. The method of claim 13, wherein the patient is non-intubated.

16. The method of claim 13, further comprising:
measuring, by a first gas detection sensor, a first concentration of the xenon in the blood prior to passing through the gas transfer unit; and
measuring, by a second gas detection sensor, a second concentration of xenon in the blood after passing through the gas transfer unit.

17. The method of claim 16, further comprising:
comparing, by a controller, the first concentration and the second concentration;
determining, by the controller when the first concentration is less than the second concentration, an additional amount of xenon gas to be delivered to the blood;
adjusting, by the controller when the first concentration is less than the second concentration, the gas transfer unit to deliver the additional amount of xenon to the blood such that the blood includes the therapeutic amount of xenon gas.

18. The method of claim 17, further comprising:
wherein the controller automatically determines the additional amount of xenon gas to be delivered to the blood.

19. The method of claim 13, further comprising:
delivering a predetermined amount of oxygen gas to the blood.

20. The method of claim 13, wherein the gas transfer unit is included in an extracorporeal membrane oxygenation (ECMO) system.

21. The method of claim 13, wherein the pump includes a centrifugal pump.

22. The method of claim 13, further comprising:
inserting a first conduit into the patient to withdraw the blood; and
inserting a second conduit into the patient to infuse the blood into the patient.

23. The method of claim 19, wherein the first conduit is operable to be inserted into and be in fluidic communication with a vein of the patient, and the second conduit is operable to be inserted into and be in fluidic communication with an artery of the patient.
